(19) United States
(12) Patent Application Publication
(10) Pub. No.: US 2023/0263925 A1
(43) Pub. Date: Aug. 24, 2023

(54) SYSTEMS AND METHODS FOR DISINFECTING AIR

(71) Applicant: THE BOEING COMPANY, CHICAGO, IL (US)

(72) Inventor: Jamie J. Childress, Mercer Island, WA (US)

(73) Assignee: THE BOEING COMPANY, CHICAGO, IL (US)

(21) Appl. No.: 17/677,385

(22) Filed: Feb. 22, 2022

(51) Int. Cl. A61L 9/20

(52) U.S. Cl. CPC A61L 9/20; A61L 2209/11; A61L 2209/12

(57) ABSTRACT

A system and method include a duct including internal reflective surfaces surrounding at least a portion of an internal air passage. One or more ultraviolet (UV) light emitters are disposed within the duct. The one or more UV light emitters are configured to emit UV light into air that passes through the internal air passage. The internal reflective surfaces reflect the UV light within the internal air passage. An air inlet is coupled to the duct. The air inlet is in fluid communication with the internal air passage. An air outlet is coupled to the duct. The air outlet is in fluid communication with the internal air passage. A blower is disposed within the duct. The blower is configured to draw the air into the internal air passage through the air inlet, and discharge the air from the internal air passage through the air outlet. The air is disinfected within the internal air passage by the UV light emitted by the one or more UV light emitters and reflected by the internal reflective surfaces.

### FIELD OF THE DISCLOSURE

Examples of the present disclosure generally relate to systems and methods for disinfecting air, such as by ultraviolet (UV) light.

### BACKGROUND OF THE DISCLOSURE

Aircraft are used to transport passengers and cargo between various locations. Passengers within an internal cabin of an aircraft can be seated in close proximity to one another.

Air within an internal cabin of an aircraft is typically a mixture of air exhaled by other passengers and fresh disinfected air. As can be appreciated, exhaled air can contain microbial particles, such as germs, bacteria, viruses, and the like.

### SUMMARY OF THE DISCLOSURE

A need exists for a system and a method for efficiently and effectively disinfecting air, such as within a confined space (for example, an internal cabin of a vehicle).

With that need in mind, certain examples of the present disclosure provide a system including a duct including internal reflective surfaces surrounding at least a portion of an internal air passage. One or more ultraviolet (UV) lights are disposed within the duct. The one or more UV lights are configured to emit UV light into air that passes through the internal air passage. The internal reflective surfaces reflect the UV light within the internal air passage. An air inlet is coupled to the duct. The air inlet is in fluid communication with the internal air passage. An air outlet is coupled to the duct. The air outlet is in fluid communication with the internal air passage. A blower is disposed within the duct. The blower is configured to draw the air into the internal air passage through the air inlet, and discharge the air from the internal air passage through the air outlet. The air is disinfected within the internal air passage by the UV light emitted by the one or more UV lights and reflected by the internal reflective surfaces.

In at least one example, the duct includes a first segment connected to a second segment through a bend. In at least one further example, the bend positions the air outlet proximate to the air inlet. For example, the bend provides a 180 degree turn.

In at least one example, the one or more UV light emitters are configured to emit the UV light at a wavelength ranging from 270-280 nanometers. In at least one other example, the one or more UV light emitters are configured to emit the UV light at a wavelength ranging from 210-260 nanometers.

In at least one example, one or both of the air inlet or the air outlet are formed of a UV absorbing material. For example, the UV absorbing material is a dark plastic.

In at least one example, the air outlet includes a nozzle. The air inlet has a first diameter, and the nozzle has a second diameter. The second diameter is less than the first diameter. In at least one example, the nozzle is moveable.

In at least one example, the duct is a light pipe.

In at least one example, the system is configured to be worn by an individual. In at least one other example, the system is incorporated into a headrest of a seat. The system can be moveable between a stowed position and a deployed position.

In at least one example, the system also includes a mounting member configured to secure the system to a structure.

In at least one example, one or both of the air inlet or the air outlet includes fins that are configured to absorb the UV light.

In at least one example, the system also includes an outlet tube extending from a nozzle of the air outlet. As a further example, the outlet tube is removably secured to the nozzle. In at least one example, the outlet tube is pivotally coupled to the nozzle.

In at least one example, one or both of the air inlet or the air outlet includes a screen.

In at least one example, the system includes a flexible tube connected to the duct. The flexible tube includes the air outlet.

Certain examples of the present disclosure provide a method including emitting, from one or more ultraviolet (UV) lights coupled to a duct including internal reflective surfaces surrounding at least a portion of an internal air passage, UV light into air that passes through the internal air passage; reflecting, by the internal reflective surfaces, the UV light within the internal air passage; drawing, by a blower coupled to the duct, the air into the internal air passage through an air inlet coupled to the duct, wherein the air inlet is in fluid communication with the internal air passage; discharging, by the blower the air from the internal air passage through an air outlet coupled to the duct, wherein the air outlet is in fluid communication with the internal air passage; and disinfecting the air is within the internal air passage by the UV light emitted by the one or more UV lights and reflected by the internal reflective surfaces.

### BRIEF DESCRIPTION OF THE DRAWINGS

FIG. 1 illustrates an isometric view of a system for disinfecting air, according to an example of the present disclosure.

FIG. 2 illustrates an internal view of the system for disinfecting air.

FIG. 3 illustrates an isometric view of the system for disinfecting air, according to an example of the present disclosure.

FIG. 4 illustrates a front view of an air inlet, according to an example of the present disclosure.

FIG. 5 illustrates a simplified internal view of the system for disinfecting air, according to an example of the present disclosure.

FIG. 6 illustrates an isometric view of the system for disinfecting air in relation to an individual, according to an example of the present disclosure.

FIG. 7 illustrates a side view of the system for disinfecting air in a stowed position on a seat, according to an example of the present disclosure.

FIG. 8 illustrates a side view of the system for disinfecting air in a deployed position on the seat.

FIG. 9 illustrates a front view of a seat having a first system for disinfecting air and a second system for disinfecting air, according to an example of the present disclosure.

FIG. 10 illustrates a simplified internal view of a headrest of a seat having the first and second systems, according to an example of the present disclosure.

FIG. 11 illustrates a top view of a pillow including a system for disinfecting air, according to an example of the present disclosure.

FIG. 12 illustrates an isometric front view of a system for disinfecting air worn by an individual, according to an example of the present disclosure.

FIG. 13 illustrates a side view of a helmet including a system for disinfecting air, according to an example of the present disclosure.

FIG. 14 illustrates an isometric front view of a headrest including systems for disinfecting air in deployed positions, according to an example of the present disclosure.

FIG. 15 illustrates an isometric front view of the headrest of FIG. 14 having the systems in stowed positions.

FIG. 16 illustrates an isometric front view of a headrest including systems for disinfecting air in deployed positions, according to an example of the present disclosure.

FIG. 17 illustrates an isometric front view of the headrest of FIG. 16 having the systems in stowed positions.

FIG. 18 illustrates an isometric front view of a headrest including systems for disinfecting air, according to an example of the present disclosure.

FIG. 19 illustrates an isometric view of a system for disinfecting air, according to an example of the present disclosure.

FIG. 20 illustrates an isometric view of a system for disinfecting air, according to an example of the present disclosure.

FIG. 21 illustrates an isometric view of a system for disinfecting air, according to an example of the present disclosure.

FIG. 22 illustrates an isometric view of a system for disinfecting air, according to an example of the present disclosure.

FIG. 23 illustrates an isometric view of a system for disinfecting air, according to an example of the present disclosure.

FIG. 24 illustrates an isometric view of a system for disinfecting air, according to an example of the present disclosure.

FIG. 25 illustrates an isometric front view of a system worn on a head of an individual, according to an example of the present disclosure.

FIG. 26 illustrates an isometric side view of the system of FIG. 25 worn on the head of the individual.

FIG. 27 illustrates an isometric rear view of the system of FIG. 25 worn on the head of the individual.

FIG. 28 illustrates a simplified internal view of a system for disinfecting air, according to an example of the present disclosure.

FIG. 29 illustrates a simplified internal view of a system for disinfecting air, according to an example of the present disclosure.

FIG. 30 illustrates a front view of an air inlet, according to an example of the present disclosure.

FIG. 31 illustrates a side view of a system for disinfecting air, according to an example of the present disclosure.

FIG. 32 illustrates an isometric front view of systems for disinfecting air, according to an example of the present disclosure.

FIG. 33 illustrates an isometric first side view of a system for disinfecting air, according to an example of the present disclosure.

FIG. 34 illustrates an isometric second side of the system of FIG. 33.

FIG. 35 illustrates an isometric front view of an individual wearing the system of FIGS. 33 and 34, according to an example of the present disclosure.

FIG. 36 illustrates a perspective view of the system of FIGS. 33 and 34 mounted to a side flap of a headrest, according to an example of the present disclosure.

FIG. 37 illustrates a flow chart of a method for disinfecting air, according to an example of the present disclosure.

### DETAILED DESCRIPTION OF THE DISCLOSURE

The foregoing summary, as well as the following detailed description of certain examples will be better understood when read in conjunction with the appended drawings. As used herein, an element or step recited in the singular and preceded by the word “a” or “an” should be understood as not necessarily excluding the plural of the elements or steps. Further, references to “one example” are not intended to be interpreted as excluding the existence of additional examples that also incorporate the recited features. Moreover, unless explicitly stated to the contrary, examples “comprising” or “having” an element or a plurality of elements having a particular condition can include additional elements not having that condition.

Air within an internal cabin of a vehicle, such as a commercial aircraft, may need additional disinfection to provide reduced active microbial particles and to increase passenger sense of well-being. Ultraviolet (UV) light can be used to neutralize microbial pathogens, such as bacteria, germs, viruses, and the like. However, shining an ultraviolet (UV) light directly on the face of an individual may not be possible at sufficient irradiance to neutralize pathogens. Examples of the present disclosure provide systems and methods that direct air flow around the face of an individual. The air flow is disinfected using UV light. One or more UV light emitters are contained in an enclosure that disinfects the air just prior to emission near the face of the individual. The UV light within the enclosure can be reflected, thereby increasing the UV exposure of the air.

In at least one example, the system includes an assembly that can be worn by an individual. As another example, the assembly can be mounted to a structure, such as a headrest of a seat. The UV light emitters can be UV light emitting diodes (LEDs), which generate minimal or low ozone. In at least one example, the system includes a UV reflective duct section supporting UV LED strips for disinfection, and UV absorbing sections, such as an at an air inlet and air outlet to prevent or otherwise reduce escape of UV light. The system can also include a blower, such as a fan, which draws air into and through the duct, and out of the air outlet. Further, the air inlet can be larger than the air outlet, thereby providing increased air velocity at the outlet (such as toward an individual’s face). The air inlet can be in close proximity to the air outlet. As such, the blower can draw air into the duct from near an individual’s face when exhaling, and the system can clean the air and provide it near the intake area.

FIG. 1 illustrates an isometric view of a system 100 for disinfecting air, according to an example of the present disclosure. The system 100 includes a duct 102 having an inlet end 104 and an outlet end 106. An air inlet 108 is disposed at the inlet end 104, and an air outlet 110 is disposed at the outlet end 106.

The duct 102 is a tube, pipe, or other such conduit that includes an outer wall 112 that defines an internal air passage 114. The internal air passage 114 provides an internal path for air to travel between the inlet end 104 and the outlet end 106, and therefore the air inlet 108 and the air outlet 110. The air inlet 108 is in fluid communication with the air outlet 110 through the internal air passage 114 of the duct 102.

As shown, the duct 102 includes a first segment 118 connected to a second segment 120 through a bend 122. The first segment 118 can be a straight, linear segment. Similarly, the second segment 120 can be a straight, linear segment. The bend 122 can provide a 180 degree turn so that the first segment 118 and the second segment 120 are generally parallel with one another. By providing a 180 degree turn, the bend 122 allows the air inlet 108 to be in close proximity to the air outlet 110. As such, the bend 122 positions the air outlet 110 proximate to the air inlet 108. For example, the air inlet 108 can be within 6 inches or less of the air outlet 110. Optionally, the duct 102 can include more bends than shown. Further, the bend 122 can be less than 180 degrees. As another example, the duct 102 may not include any bend. Instead, the air inlet 108 and the air outlet 110 can be at opposite ends of a straight duct.

One or more ultraviolet (UV) light emitters 124 (or UV lights) are coupled to a duct 102. For example, the UV light emitters 124 can be disposed within the duct 102. As another example, at least portions of the UV light emitters 124 can be outside of the duct 102. As an example, the UV light emitters 124 can protrude into openings formed in the duct 102. The UV light emitters 124 can be secured to portions of the duct 102 through one or more fasteners, adhesives, or the like. For example, a plurality of UV light emitters 124 are disposed within the internal air passage 114 within the first segment 118, and a plurality of UV light emitters 124 are disposed within the internal passage 114 within the second segment 118. Optionally, UV light emitters 124 can be disposed within the bend 122. As another example, one or more UV light emitters 124 are disposed within the one of the first segment 118, the second segment 120, or the bend 122. As another example, one or more UV light emitters 124 are disposed within each of the first segment 118, the second segment 120, and the bend 122.

The UV light emitters 124 are configured to emit UV light into air that passes through the duct 102, thereby disinfecting the air as it passes from the air inlet 108 and to and through the air outlet 110. In at least one example, the UV light emitters 124 are configured to emit UV light at a wavelength ranging from 270-280 nanometers (nm). Optionally, the UV light emitters 124 can be configured to emit UV light at different wavelengths, such as ranging from 210-230 nm, 240-260 nm, and/or the like.

The duct 102 is formed of (or has internal portions formed of or coated with) a reflective material. For example, the duct 102 is formed of aluminum. In another example, the duct 102 can be formed of Teflon. Internal surfaces of the duct 102 that define the internal air passage 114 are formed of, or otherwise coated, with a reflective material, such as aluminum, or Teflon. Outer surfaces of the outer wall 112 are formed of, or otherwise coated with an opaque material, such as a metal, thereby ensuring that UV light emitted by the UV light emitters 124 does not escape out and through the outer wall 112 of the duct 102. In this manner, the duct 102 is a light pipe that internally reflects UV light emitted by the UV light emitters 124 but prevents the UV light from escaping through the outer wall 112.

The air inlet 108 and the air outlet 110 are formed of UV absorbing material. For example, the air inlet 108 and the air outlet 110 are formed of a dark plastic, which absorbs UV light, thereby eliminating, minimizing, or otherwise reducing the potential of UV light escaping therethrough. For example, the air inlet 108 and the air outlet 110 can be formed of dark, opaque plastic. In at least one example, the air inlet 108 and the air outlet 110 can be black plastic. The darker the plastic, the more UV light will be absorbed.

A blower 126, such as a fan, is coupled to the duct 102. For example, the blower 126 can be disposed within the duct 102. As another example, the blower 126 can have a portion disposed within the duct 102, and another portion outside of the duct 102. As another example, the duct 102 can have an opening into which a conduit that connects to the blower 126 is secured. The blower 126 can be secured within the duct 102 through one or more fasteners, adhesives, and/or the like. As an example, the blower 126 is disposed within the bend 122. Optionally, the blower 126 can be disposed within the first segment 118 or the second segment 120. As another example, additional blowers 126 can be disposed within one or more portions of the duct 102.

The air inlet 108 has a first diameter 130 that defines an opening through which air is drawn into the duct 102. The air outlet 110 has a conic body 132 having a nozzle 133 defining a second diameter 134, which is substantially smaller than the first diameter 130. The second diameter 134 defines an opening through which air is discharged from the system 100. For example, the second diameter 134 can be half or less than the first diameter 130. As another example, the second diameter 134 is a quarter or less than the first diameter 130. By reducing the size of the second diameter 134 of the air outlet 110 in relation to the first diameter 130 of the air inlet 108, air discharged through the air outlet 110 is at increased velocity as compared to air that is drawn in through the air inlet 108. Optionally, the second diameter 134 of the air outlet 110 may not be substantially smaller than the first diameter 130 of the air inlet 108. For example, the first diameter 130 and the second diameter 134 can be alternatively equal to one another.

In at least one example, a support insert 139, such as a bracket, block, or the like, is secured between the first segment 118 and the second segment 120. The support insert 139 ensures that the first segment 118 and the second segment 120 do not undesirably encroach upon one another. Optionally, the system 100 may not include the support insert 139.

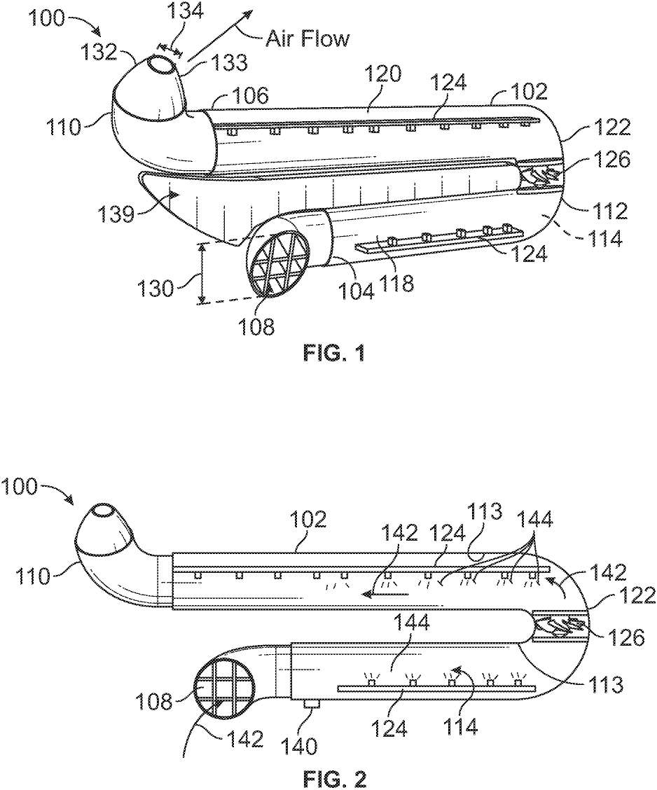

FIG. 2 illustrates an internal view of the system 100 for disinfecting air. Referring to FIGS. 1 and 2, the system 100 can include an activation switch 140 that is in communication with the UV light emitters 124 and the blower 126, such as through one or more wired or wireless connections. The activation switch 140 can be mounted on and/or within the duct 102, the air inlet 108, or the air outlet 110. Optionally, the activation switch 140 can be remotely located from the duct 102, the air inlet 108, or the air outlet 110. For example, the activation switch 140 can be mounted to a portion of a seat.

When the switch 140 is in an ON position, the UV light emitters 124 are activated to emit the UV light, and the blower 126 is activated to draw air into the duct 102 through the air inlet 108, and out through the air outlet 110. When the switch 140 is in an OFF position, the UV light emitters 124 and the blower 126 are deactivated. The switch 140 can be a or otherwise include a physical switch, such as a button, key, dial, toggle, or the like that is configured to be selectively engaged by an individual between the ON and OFF positions. Optionally, the switch 140 can be or include a sensor that is configured to automatically activate and deactivate the UV light emitters 124 and the blower 126. For example, the sensor can be a motion or fluid sensor that detects individual motion, fluid flow, and/or the like.

In operation, the blower 126 is activated to draw air 142 into the duct 102 through the air inlet 108. The blower 126 can be configured to move the air 142 within the internal air passage 114 of the duct 102 at a relatively low velocity to ensure that the air 142 is exposed to the UV light 144 for a sufficient amount of time to disinfect the air 142. The smaller diameter 134 of the air outlet 110 ensures that the disinfected air 142 is expelled at a higher velocity than air is drawn in through the air inlet 108. As such, the reduced diameter nozzle 133 increases the velocity of disinfected air that is expelled out of the system 100 (such as onto a face of an individual). At the same time, the larger diameter of the internal air passage 114 limits the velocity of air flowing therein, which increases the amount of time the air 142 is exposed to the UV light 144 emitted and reflected within the duct 102. As an example, the diameter of the air inlet 108 and the internal air passage 114 can range from 1 – 2 inches, while the diameter of the nozzle 133 can range from 0.1-0.5 inches.

As the air passes through the internal air passage 114, the UV light emitters 124 emit UV light 144 into the flowing air 142, thereby disinfecting the air 142. The emitted UV light 144 internal reflects off the internal reflective surfaces of the duct 102 (such as a light pipe), thereby continually passing into and through the air 142, which provides increased and efficient disinfection of the air 142. The UV light within the duct 102 is continually internally reflected, thereby increasing the air to increased UV exposure. The blower 126 moves the air through the internal air passage 114 toward the air outlet 110, with the UV light emitters 124 emitting the UV light 144 into the air 142 between the air inlet 108 and the air outlet 110 to disinfect the air 142 (for example, neutralize microbial pathogens, such as germs, bacteria, viruses, and the like). Because the air inlet 108 and the air outlet 110 are formed of UV absorbing material (such as a dark plastic), the potential of UV light escaping out of the system 100 is eliminated, minimized, or otherwise reduced.

The duct 102 provides a path for air to pass through, and be disinfected by UV light emitted from the UV light emitters 124 and internally reflected within the duct 102. The duct 102 can provide a circuitous path that ensures that the air 142, as moved by the blower 126, is exposed to UV light for a sufficient amount of time to disinfect the UV light (for example, neutralize any pathogens contained therein). The air 142 is disinfected by the UV light emitted by the UV light emitters 124 before being discharged through the air outlet 110.

In at least one example, the UV light 144 emitted by the UV light emitters 124 is selected to have low ozone emission into an air stream (for example, UV LEDs that emit UV light at a wavelength of 222 nm). Any UV light that escapes the system 100 is sufficiently low that long duration passenger exposure is within allowable limits as defined by regulatory agencies (such as the Federal Aviation Administration). The UV light 144 within the duct 102 is reflected many times from internal reflective surfaces 113 that define the internal air passage 114, thereby increasing the UV exposure of the air. For example, the internal reflective surfaces 113 can be formed of or otherwise coated with Teflon, which has approximately 96% reflectivity at UV frequencies allowing high UV irradiance along the internal air passage 114.

The bend 122 disposes the air inlet 108 proximate to the air outlet 110. As such, both the air inlet 108 and the air outlet 110 can be disposed close to a face of an individual, thereby ensuring the air exhaled by an individual is drawn into the duct 102, disinfected as described herein, and expelled for the induvial to breathe.

The system 100 provides disinfected air while consuming less power as compared to a UV light that is configured to direct UV light directly onto a face of an individual. Further, the system 100 eliminates, minimizes, or otherwise reduces UV exposure to skin and eyes of an individual. The system 100 also provides increased disinfection of air next to an individual’s face. Also, the blower 126 is configured to consume a relatively small amount of power and move air at a relatively low velocity, thereby decreasing operational costs, and reducing generated noise.

The system 100 can be worn by an individual. For example, the system 100 can include a clip, hook, loop, or the like that allow an individual to wear the system 100. As another example, the system 100 can be secured to a structure, such as headrest of a seat, such as within a vehicle (for example, a commercial aircraft), or within a venue such as a theater, stadium, or the like.

As described herein, the system 100 includes the duct 102 including the internal reflective surfaces 113 surrounding at least a portion of the internal air passage 114. One or more ultraviolet (UV) lights 124 are disposed within the duct 102. The one or more UV lights 124 are configured to emit UV light 144 into the air 142 that passes through the internal air passage 114. The internal reflective surfaces 113 reflect the UV light 144 within the internal air passage 114. The air inlet 108 is coupled to the duct, 102. The air inlet 108 is in fluid communication with the internal air passage 114. The air outlet 110 is coupled to the duct 102. The air outlet 110 is in fluid communication with the internal air passage 114. The blower 126 is disposed within the duct 102. The blower 126 is configured to draw air 142 into the internal air passage 114 through the air inlet 108, and discharge the air 142 from the internal air passage 114 through the air outlet 110. The air 142 is disinfected within the internal air passage 114 by the UV light 144 emitted by the one or more UV lights 124 and reflected by the internal reflective surfaces 113.

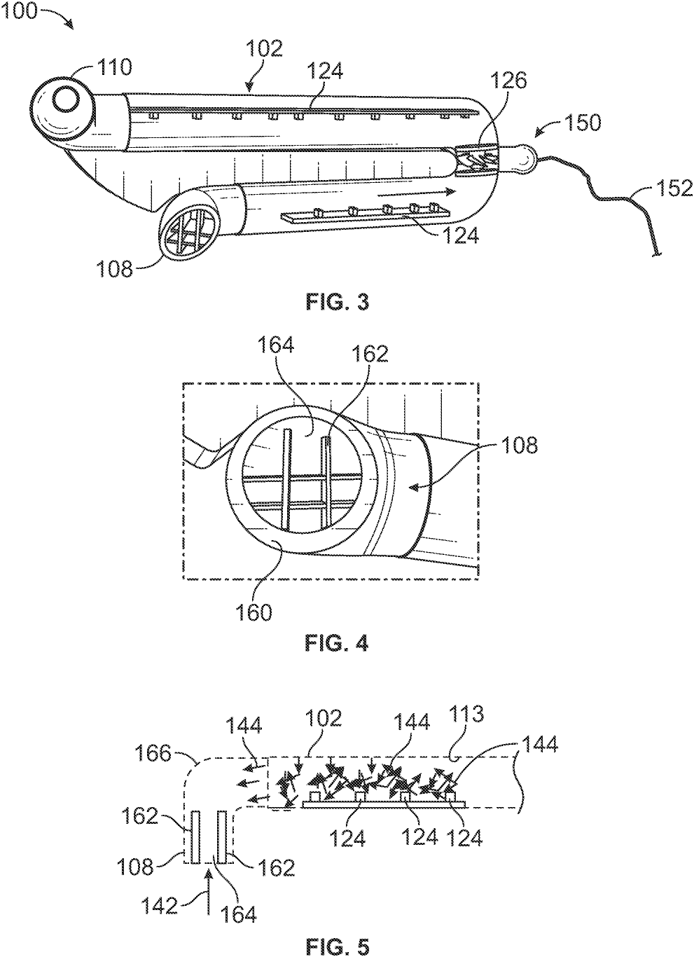

FIG. 3 illustrates an isometric view of the system 100 for disinfecting air, according to an example of the present disclosure. As shown, the system 100 can include a mounting member 150, which is configured to secure the system 100 to a structure, such as headrest of a seat. The mounting member 150 can be a clamp, bracket, or the like. In at least one example, the mounting member 150 is or otherwise includes a ball pivot, hinge, swivel, or the like. A power line 152 (such as a cable or wire) connects to the blower 126 and the UV light emitters 124. The power line 152 provides electrical power from a power source to the blower 126 and the UV light emitters 124. Optionally, the system 100 can include an internal source of power, such as one or more batteries.

FIG. 4 illustrates a front view of the air inlet 108, according to an example of the present disclosure. In at least one example, the air inlet 108 includes a plurality of intersecting fins 162 disposed within an internal channel 164. The fins 162 can be flat panels formed of a UV light absorbing material, such as a plastic. Air is drawn into the air inlet 108 through the internal channel 164, while the fins 162 provide an additional barrier that absorbs UV light and prevents the UV light from passing out of the air inlet 108. The air outlet 110 (shown in FIGS. 1-3) can also include fins as shown and described with respect to FIG. 4. The fins 162 provide low air flow resistance, and high UV light absorption.

FIG. 5 illustrates a simplified internal view of the system for 100 disinfecting air, according to an example of the present disclosure. The air inlet 108 can include a bend 166 that leads to the duct 102. The fins 162 extend into the air inlet 108 toward the bend 166. The fins 162 provide barriers that block UV light 144 from escaping through the air inlet 108. As the UV light 144 impinges on the fins 162, the UV light is absorbed by the fins 162. The air 142 is drawn in through the air outlet and into the duct 102, such as by the blower 126 (shown in FIGS. 1-3).

As shown, the internal reflective surfaces 113 of the duct 102 internally reflect the UV light 144 emitted by the UV light emitters 124. Accordingly, the duct 102 provides a light pipe that is configured to internally reflect the UV light 144.

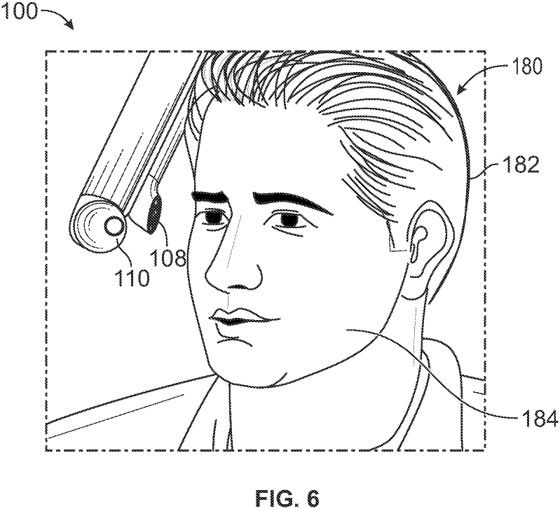

FIG. 6 illustrates an isometric view of the system 100 for disinfecting air in relation to an individual 180, according to an example of the present disclosure. In at least one example, the individual 180 wears the system 100. For example, the system 100 can include a clip, hook, loop, or the like that allows the system 100 to be worn on a head 182. As another example, the system 100 can be secured to a headrest of a seat on which the individual 180 is seated.

As described above, the air inlet 108 and the air outlet 110 can be in closed proximity to one another. Referring to FIGS. 1-6, the bend 122 orients the first segment 118 and the segment 120 to be substantially parallel (such as within 5 degrees), which allows the air inlet 108 to be in close proximity (such as within 6 inches) to the air outlet 110.

The UV light emitters 124 emit the UV light 144, which allows local air surrounding the individual 180 to be disinfected within the duct 102, which provides a circuitous loop and light pipe. The blower 126 draws air from near the face 184 of the individual in through the air inlet 108. The air is disinfected by UV light 144 emitted by the UV light emitters 124 and internally reflected within the duct 102. The disinfected air is then discharged through the air outlet 110 near the face 184. Because the disinfection of the air is highly localized, even aerosolized pathogens from an individual sitting next to the individual 180 will be neutralized.

FIG. 7 illustrates a side view of the system 100 for disinfecting air in a stowed position on a seat 190, according to an example of the present disclosure. Referring to FIGS. 1-3 and 7, the mounting member 150 secures the system 100 to a portion of the seat 190, such as to a side portion of a headrest 192. The mounting member 150 can be a pivoting structure, such as a ball pivot, which allows the system 100 to rotate about an axis.

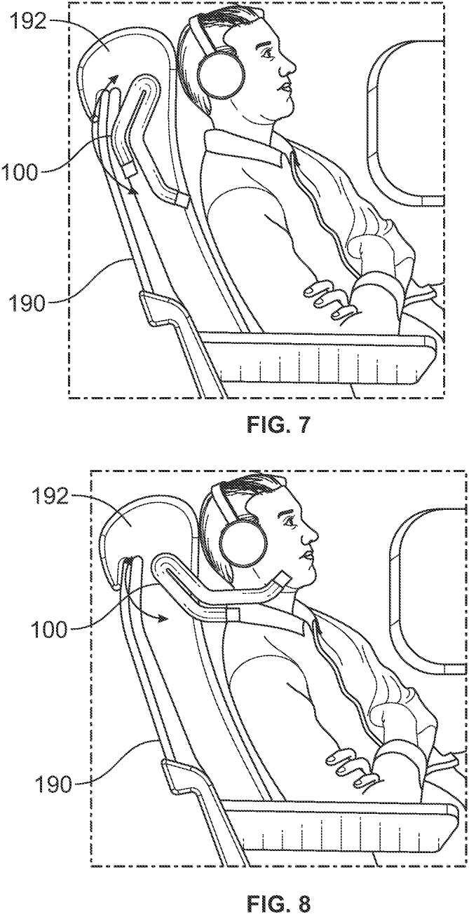

FIG. 8 illustrates a side view of the system 100 for disinfecting air in a deployed position on the seat 190. An individual can pivot the system 100 between the stowed position (shown in FIG. 7) and the deployed position, such as about the mounting member 150.

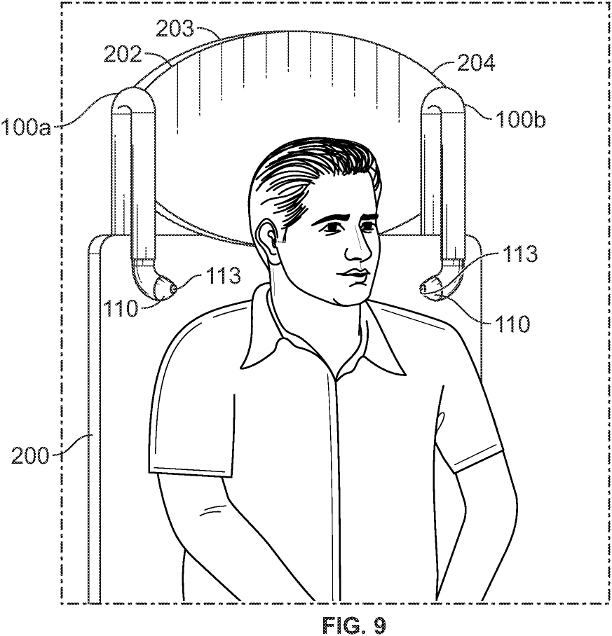

FIG. 9 illustrates a front view of a seat 200 having a first system 100a for disinfecting air and a second system 100a for disinfecting air, according to an example of the present disclosure. The first system 100a and the second system 100b are configured as any of the systems 100 described herein. The first system 100a is secured to a first side 202 of a headrest 203, and the second system 100b is secured to a second side 204 of the headrest 203. The second side 204 is opposite from the first side 202. The first system 100a and the second system 100b can be fixed in position. Optionally, the first system 100a and the second system 100b can be moveably coupled to the headrest 203 and configured to be moved between stowed and deployed positions.

In at least one example, the air outlets 110 can be fixed in position. In at least one other example, the nozzle 133 is movable. For example, the nozzle 133 can be pivotally mounted to allow for rotation to desired positions. Any of the examples described herein can include moveable nozzles 133.

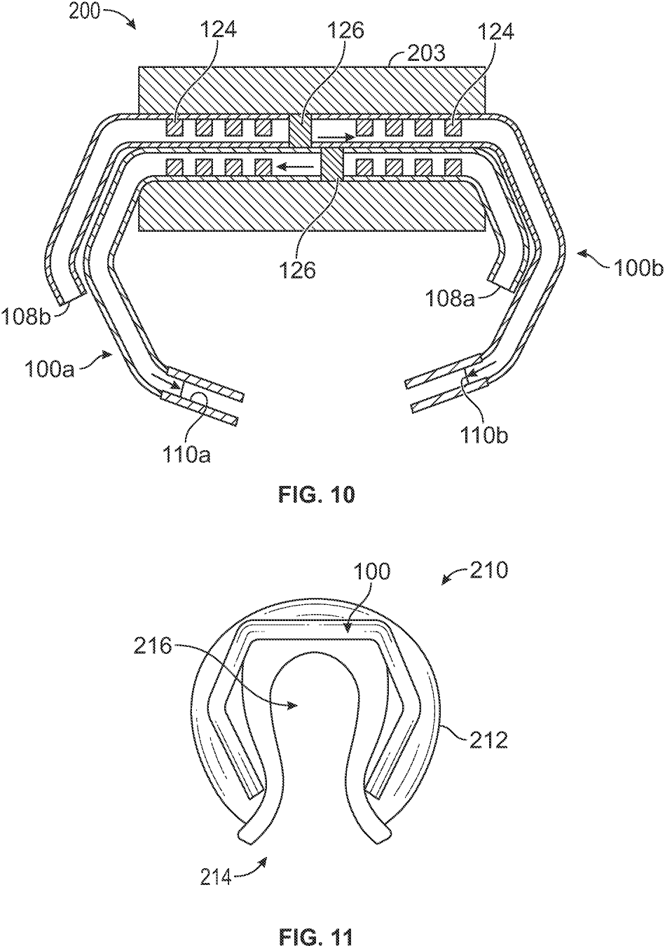

FIG. 10 illustrates a simplified internal view of the headrest 203 of the seat 200 having the first and second systems 100a and 100b, according to an example of the present disclosure. As shown, ducts 102 of the systems 100a and 100b can pass through internal portions of the headrest 203. The ducts 102 can be fixed within the headrest 203.

FIG. 11 illustrates a top view of a pillow 210 including the system 100 for disinfecting air, according to an example of the present disclosure. The pillow 210 can be a neck pillow having an arcuate main body 212 defining an opening 214 that leads into a neck cavity 216. The pillow 210 can be configured to be worn around a neck of an individual. The system 100, such as any of those described herein, can be incorporated into the pillow 210. For example, the system 100 can be mounted on a portion of the main body 212. As another example, the system 100 can be disposed within at least a portion of the main body 212. Accordingly, the system 100 can be incorporated into the pillow 210.

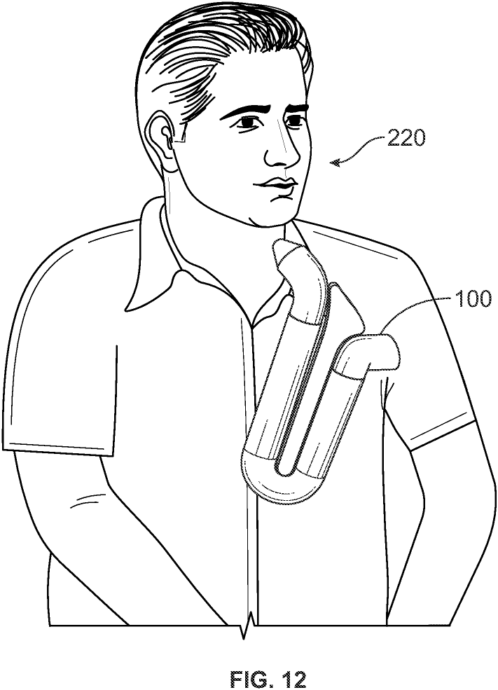

FIG. 12 illustrates an isometric front view of the system 100 for disinfecting air worn by an individual 220, according to an example of the present disclosure. The system 100, such as any of those described herein, can include one or more straps, hooks, loops, or the like that allow the individual 220 to wear the system 100 over a chest. In another example, the system 100 can be worn on a head of the individual.

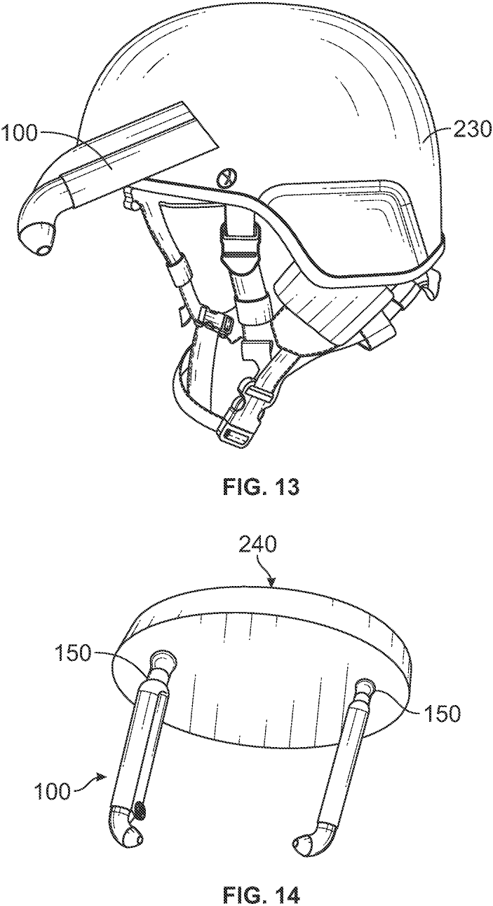

FIG. 13 illustrates a side view of a helmet 230 including a system 100 for disinfecting air, according to an example of the present disclosure. The system 100, such as any of those described herein, can be secured to a portion of the helmet 230, such as an outer or interior portion of the helmet 230. As such, the system 100 can be incorporated into the helmet 230.

FIG. 14 illustrates an isometric front view of a headrest 240 including systems 100a and 100b for disinfecting air in deployed positions, according to an example of the present disclosure. The systems 100a and 100b can be configured as any of the systems 100 described herein. The systems 100a and 100b moveably couple to the headrest 240 through the mounting members 150, which can be pivot joints.

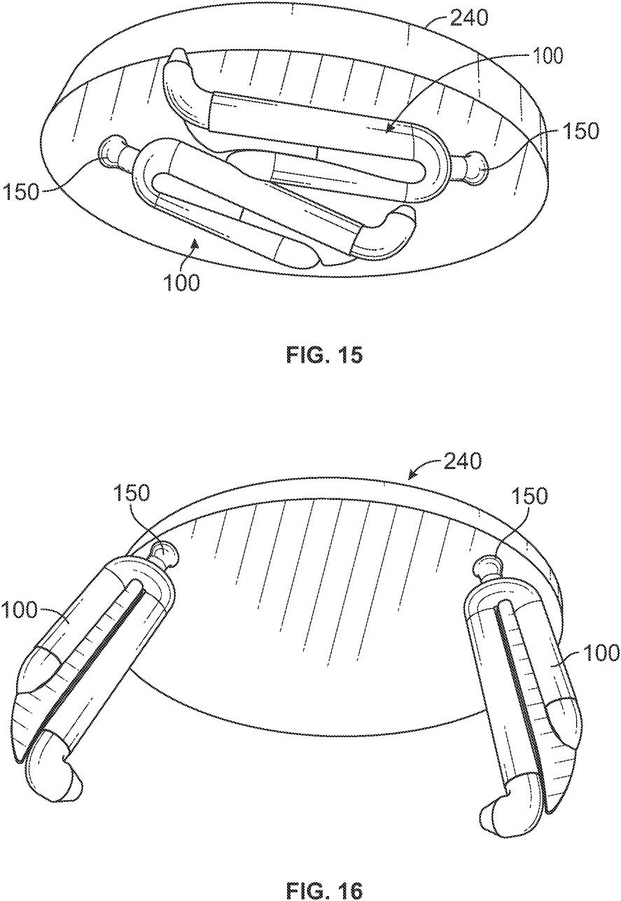

FIG. 15 illustrates an isometric front view of the headrest 240 of FIG. 14 having the systems in stowed positions. As shown, the systems 100a and 100b can outwardly pivot to deployed positions, as shown in FIG. 14, and inwardly pivot across the headrest 203 to stowed positions.

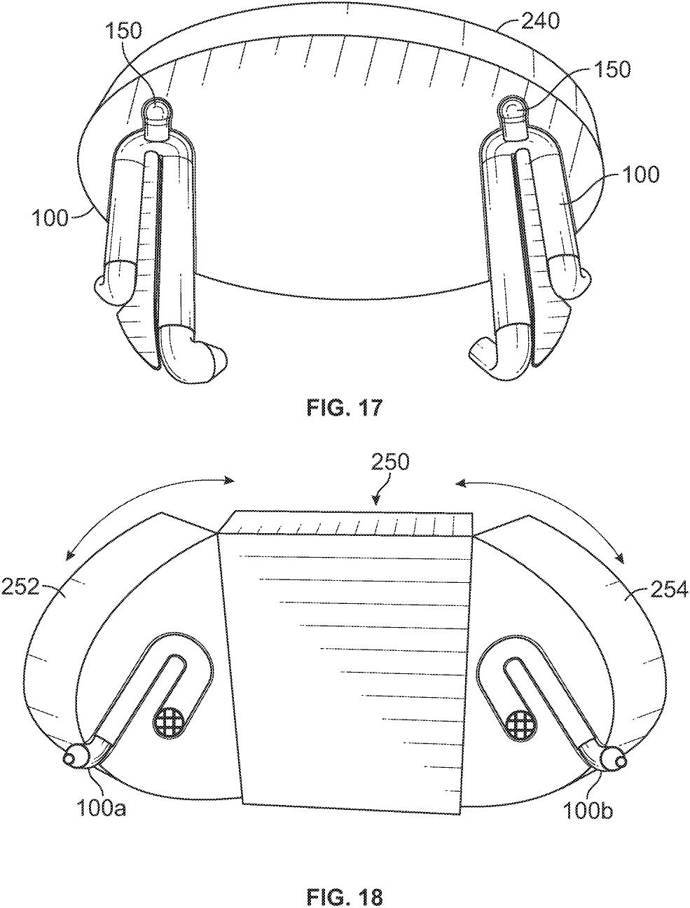

FIG. 16 illustrates an isometric front view of a headrest 240 including systems 100a and 100b for disinfecting air in deployed positions, according to an example of the present disclosure. FIG. 17 illustrates an isometric front view of the headrest 240 of FIG. 16 having the systems 100a and 100b in stowed positions. The headrest 240 shown in FIGS. 16 and 17 is similar to that shown in FIGS. 14 and 15, except that the systems 100a and 100b can be configured to downwardly pivot into the stowed positions, and upwardly pivot into the deployed positions.

FIG. 18 illustrates an isometric front view of a headrest 250 including systems 100a and 100b for disinfecting air, according to an example of the present disclosure. The systems 100a and 100b can be configured as any of the systems 100 described herein. As shown, the systems 100a and 100b can be integrated into moveable side flaps 252 and 254 of the headrest 250.

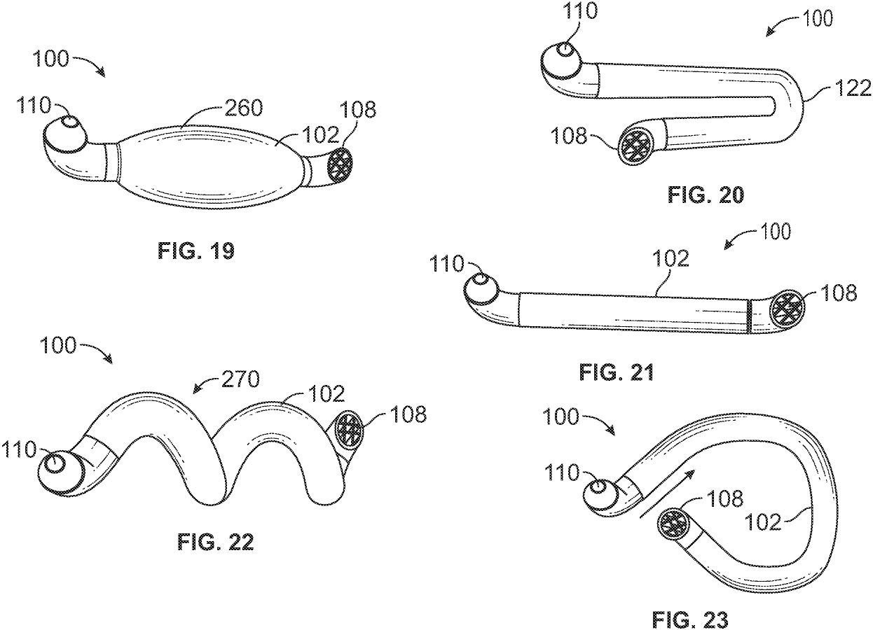

FIG. 19 illustrates an isometric view of a system 100 for disinfecting air, according to an example of the present disclosure. As shown, the duct 102 may not include a bend. Instead, the air outlet 110 can be distally located from the air inlet 108. The duct 102 can also include an expanded main body 260, which has a larger diameter than the air inlet 108 and the air outlet 110. The expanded main body 260 provides a larger UV irradiance zone, which increases air disinfection for a given input power. Any of the examples described herein can include a duct having an expanded main body 260, such as shown in FIG. 19. For example, the first and second segments of the duct 102 shown in FIG. 1 can include expanded main bodies.

FIG. 20 illustrates an isometric view of a system 100 for disinfecting air, according to an example of the present disclosure. As shown, the duct 102 can include the bend 122. The duct 102 can include additional bends.

FIG. 21 illustrates an isometric view of a system 100 for disinfecting air, according to an example of the present disclosure. As shown, the duct 102 can include a straight main body that has a diameter that is the same, or substantially the same, as the air inlet 108.

FIG. 22 illustrates an isometric view of a system for disinfecting air, according to an example of the present disclosure. As shown, the duct 102 can include a spiraled main body 270, which provides a longer path for air to travel and thereby be exposed to UV light therein.

FIG. 23 illustrates an isometric view of a system 100 for disinfecting air, according to an example of the present disclosure. As shown, the duct 102 can have an irregularly-curved shape. The duct 102 can be sized and shaped as desired.

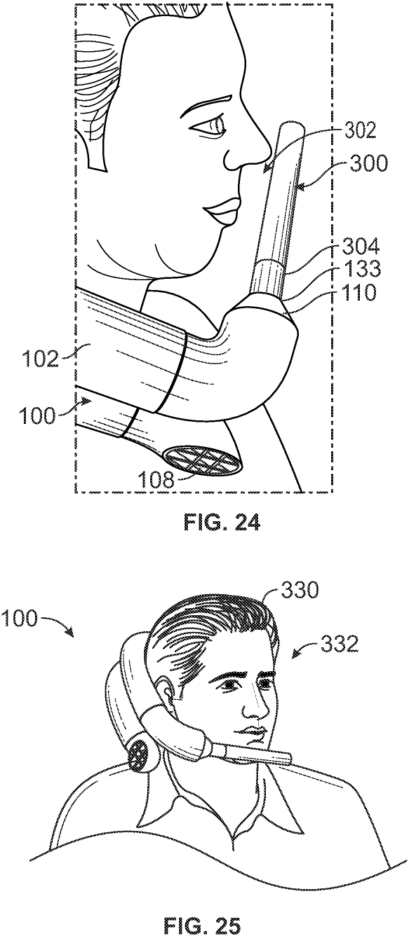

FIG. 24 illustrates an isometric view of a system 100 for disinfecting air, according to an example of the present disclosure. As shown, an outlet tube 300 (for example, a flexible hose) can extend from the nozzle 133 of the air outlet 110. The outlet tube 300 be permanently secured to the nozzle 133. Optionally, the outlet tube 300 can be removably secured to the nozzle 133, such as via a threadable interface, a snapable interface, an interference fit, or the like. In this manner, the outlet tube 300 can be a disposable tube that can be removably coupled to the air outlet 110.

The outlet tube 300 includes one or more air openings 302, such as a linear slot, which allow air to be expelled therefrom. A pivot joint 304 can couple the outlet tube 300 to the nozzle 133. The pivot joint 304 allows an individual to selectively adjust and orient the outlet tube 300, as desired. The pivot joint 304 allows an individual to control the direction of disinfected air.

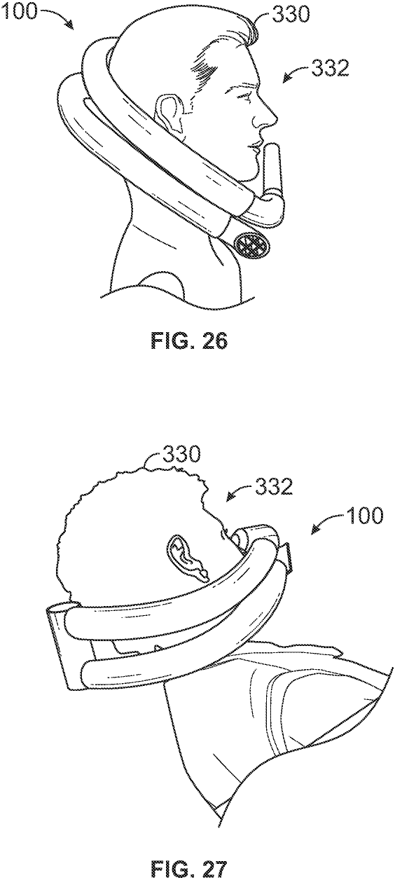

FIG. 25 illustrates an isometric front view of a system 100 worn on a head 330 of an individual 332, according to an example of the present disclosure. FIG. 26 illustrates an isometric side view of the system 100 of FIG. 25 worn on the head 330 of the individual 332. FIG. 27 illustrates an isometric rear view of the system 100 of FIG. 25 worn on the head 330 of the individual 332. The system 100 can be configured as any of those described herein. The system 100 can include one or more straps, loops, hooks, or the like that allow the system 100 to be worn on the head 330. In at least one example, the system 100 can be incorporated into a hood, headband, or the like that is worn by the individual 332. As another example, the system 100 can be supported by shoulders of the individual 332. As another example, the system 100 can partially loop around the head and/or neck of the individual 332.

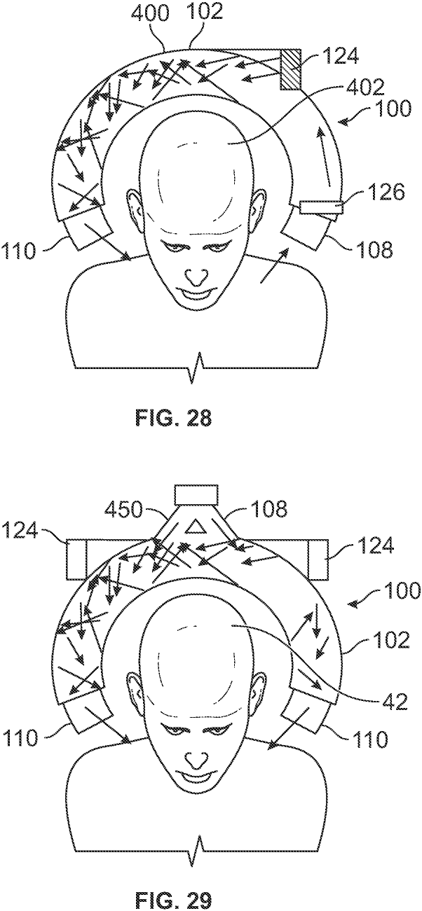

FIG. 28 illustrates a simplified internal view of a system 100 for disinfecting air, according to an example of the present disclosure. The duct 102 can have an arcuate main body 400 that is configured to extend around a portion of a head 402. The UV light emitter(s) 124 can be disposed within any portion of the duct 102. The blower 126 can be proximate to the air inlet 108. Optionally, the blower 126 can be within any other portion of the duct 102.

FIG. 29 illustrates a simplified internal view of a system 100 for disinfecting air, according to an example of the present disclosure. In this example, the air inlet 108 can be proximate to a middle section 450 of the duct 102 behind the head 402. The duct 102 further includes two air outlets 110 at opposite ends that are configured to be proximate to opposite sides of the head 402. The blower 126 can be disposed proximate to the air inlet 108. Multiple UV light emitters 124 can be used.

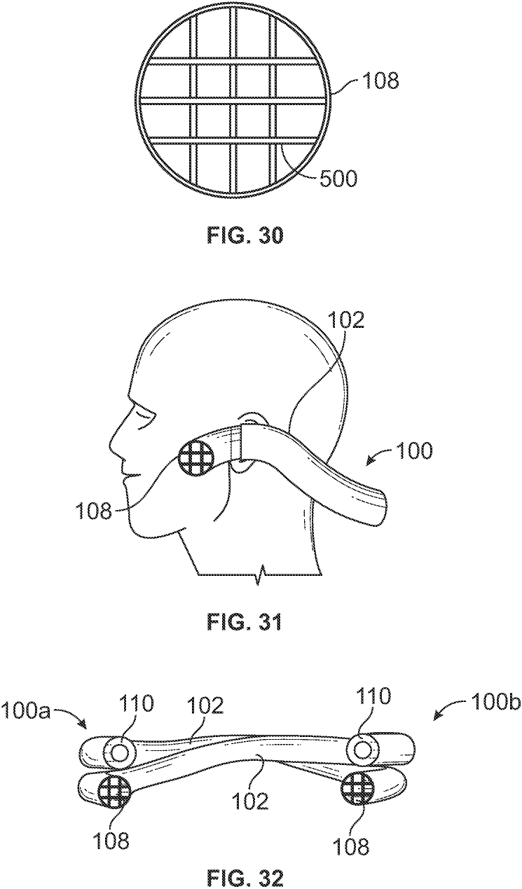

FIG. 30 illustrates a front view of the air inlet, 108 according to an example of the present disclosure. The air inlet 108 can include a screen 500, such as a metal mesh screen, disposed therein. The air outlet 110 can also include a screen 500. The screen 500 prevents foreign object debris from passing into the air inlet 108 (and/or the air outlet 110).

FIG. 31 illustrates a side view of a system 100 for disinfecting air, according to an example of the present disclosure. As shown, the duct 102 can be sized to rise above a height of the air inlet 108 (or optionally, the air outlet 110) to prevent foreign object debris from passing into the duct 102.

FIG. 32 illustrates an isometric front view of systems 100a and 100b for disinfecting air, according to an example of the present disclosure. The systems 100a and 100b can be configured as any of those described herein. The systems 100a and 100b are configured to wrap around a head of an individual. The ducts 102 of the systems 100a and 100b are shaped such that the air inlet 108 of the system 100a is below the air outlet 110 of the system 100b, and the air inlet 108 of the system 100b is below air outlet 110 of the system 100a.

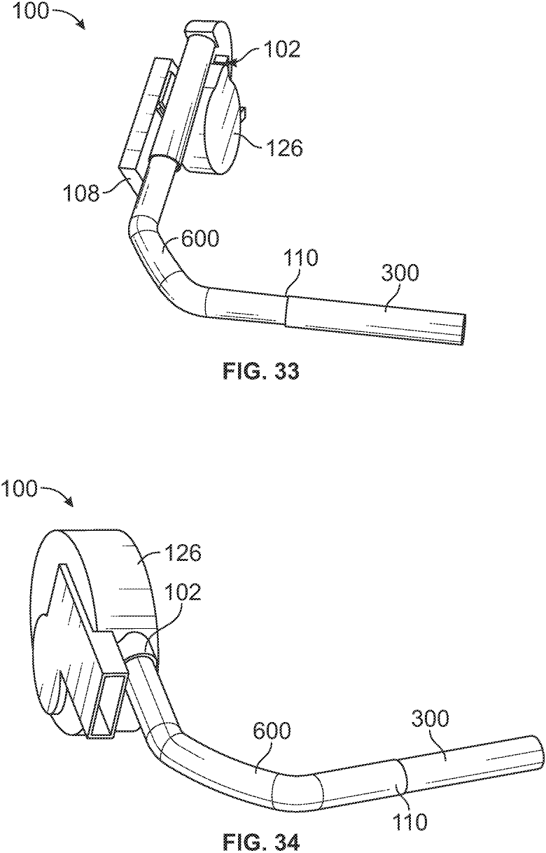

FIG. 33 illustrates an isometric first side view of a system 100 for disinfecting air, according to an example of the present disclosure. FIG. 34 illustrates an isometric second side of the system 100 of FIG. 33. As shown, the air inlet 108 is mounted to a side of the blower 126 and the duct 102, which provides the light pipe, as described herein. A flexible tube 600 extends from the duct 102, and provides the air outlet 110. An outlet tube 300 can extend from the air outlet 110, as described herein. Optionally, the flexible tube 300 can be a rigid conduit, such as a solid pipe.

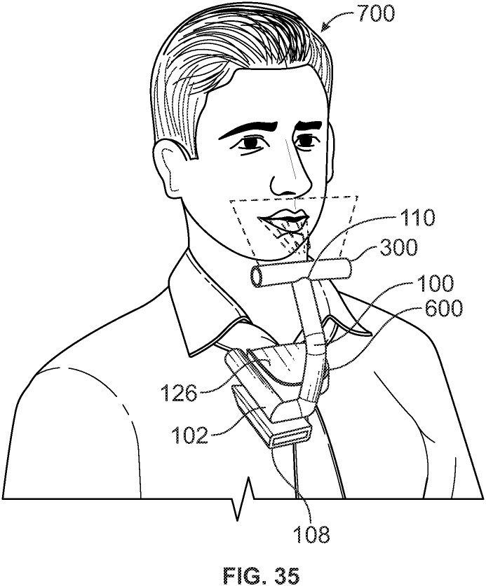

FIG. 35 illustrates an isometric front view of an individual 700 wearing the system 100 of FIGS. 33 and 34, according to an example of the present disclosure. The system 100 can include one or more features that allow the individual 700 to wear the system 100, as described herein. Optionally, the system 100 can be incorporated into a structure, such as a headrest, pillow, helmet, or the like.

The flexible tube 600 allows the individual 700 to move the outlet tube 300 to a desired position. Further, the outlet tube 300 can be pivoted, extended, and/or the like in relation to the air outlet 110.

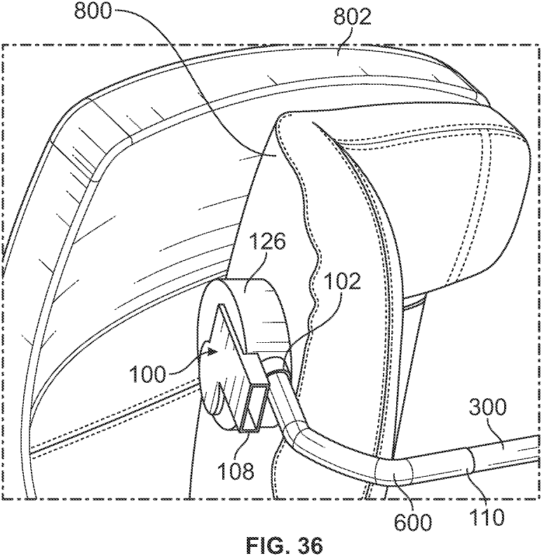

FIG. 36 illustrates a perspective view of the system 100 of FIGS. 33 and 34 mounted to a side flap 800 of a headrest 802, according to an example of the present disclosure. As shown, the air inlet 108, the blower 126, and the duct 102 can be secured behind the side flap 800, and the flexible tube 600 extends in front of the side flap 800.

In at least one example, the systems and methods described herein are maskless. That is, the systems 100 do not include a mask that is worn around and over a mouth and/or nose of an individual. Such maskless systems do not visually restrict an individual. Alternatively, the air inlet 108 and/or the air outlet 110 can be in communication with a mask that is worn over a portion of the face.

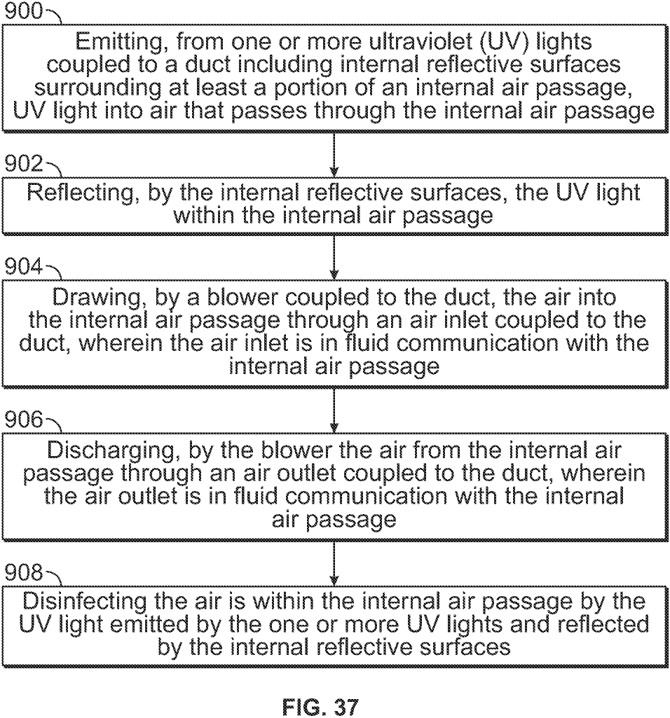

FIG. 37 illustrates a flow chart of a method for disinfecting air, according to an example of the present disclosure. The method includes emitting 900, from one or more ultraviolet (UV) lights coupled to a duct including internal reflective surfaces surrounding at least a portion of an internal air passage, UV light into air that passes through the internal air passage; reflecting 902, by the internal reflective surfaces, the UV light within the internal air passage; drawing 904, by a blower coupled to the duct, the air into the internal air passage through an air inlet coupled to the duct, wherein the air inlet is in fluid communication with the internal air passage; discharging 906, by the blower the air from the internal air passage through an air outlet coupled to the duct, wherein the air outlet is in fluid communication with the internal air passage; and disinfecting 908 the air is within the internal air passage by the UV light emitted by the one or more UV lights and reflected by the internal reflective surfaces.

Further, the disclosure comprises examples according to the following clauses:

Clause 1. A system comprising:
a duct including internal reflective surfaces surrounding at least a portion of an internal air passage;
one or more ultraviolet (UV) lights coupled to the duct, wherein the one or more UV lights are configured to emit UV light into air that passes through the internal air passage, wherein the internal reflective surfaces reflect the UV light within the internal air passage;
an air inlet coupled to the duct, wherein the air inlet is in fluid communication with the internal air passage;
an air outlet coupled to the duct, wherein the air outlet is in fluid communication with the internal air passage; and
a blower coupled to the duct, wherein the blower is configured to draw the air into the internal air passage through the air inlet, and discharge the air from the internal air passage through the air outlet, and wherein the air is disinfected within the internal air passage by the UV light emitted by the one or more UV lights and reflected by the internal reflective surfaces.

Clause 2. The system of Clause 1, wherein the duct comprises a first segment connected to a second segment through a bend.

Clause 3. The system of Clause 2, wherein the bend positions the air outlet proximate to the air inlet.

Clause 4. The system of Clauses 2 or 3, wherein the bend provides a 180 degree turn.

Clause 5. The system of any of Clauses 1-4, wherein the one or more UV light emitters are configured to emit the UV light at a wavelength ranging from 270-280 nanometers.

Clause 6. The system of any of Clauses 1-5, wherein the one or more UV light emitters are configured to emit the UV light at a wavelength ranging from 210-260 nanometers.

Clause 7. The system of any of Clauses 1-6, wherein one or both of the air inlet or the air outlet are formed of a UV absorbing material.

Clause 8. The system of Clause 7, wherein the UV absorbing material is a dark plastic.

Clause 9. The system of any of Clauses 1-8, wherein the air outlet comprises a nozzle, wherein the air inlet has a first diameter, and the nozzle has a second diameter, and wherein the second diameter is less than the first diameter.

Clause 10. The system of Clause 9, wherein the nozzle is moveable.

Clause 11. The system of any of Clauses 1-10, wherein the duct is a light pipe.

Clause 12. The system of any of Clauses 1-11, wherein the system is configured to be worn by an individual.

Clause 13. The system of any of Clauses 1-11, wherein the system is incorporated into a headrest of a seat.

Clause 14. The system of Clause 13, wherein the system is moveable between a stowed position and a deployed position.

Clause 15. The system of any of Clauses 1-14, further comprising a mounting member configured to secure the system to a structure.

Clause 16. The system of any of Clauses 1-15, wherein one or both of the air inlet or the air outlet comprises fins that are configured to absorb the UV light.

Clause 17. The system of any of Clauses 1-16, further comprising an outlet tube extending from a nozzle of the air outlet.

Clause 18. The system of Clause 17, wherein the outlet tube is removably secured to the nozzle.

Clause 19. The system of Clauses 17 or 18, wherein the outlet tube is pivotally coupled to the nozzle.

Clause 20. The system of any of Clauses 1-19, wherein one or both of the air inlet or the air outlet comprises a screen.

Clause 21. The system of any of Clauses 1-20, further comprising a flexible tube connected to the duct, wherein the flexible tube comprises the air outlet.

Clause 22. A method comprising:
emitting, from one or more ultraviolet (UV) lights coupled to a duct including internal reflective surfaces surrounding at least a portion of an internal air passage, UV light into air that passes through the internal air passage;
reflecting, by the internal reflective surfaces, the UV light within the internal air passage;
drawing, by a blower coupled to the duct, the air into the internal air passage through an air inlet coupled to the duct, wherein the air inlet is in fluid communication with the internal air passage;
discharging, by the blower the air from the internal air passage through an air outlet coupled to the duct, wherein the air outlet is in fluid communication with the internal air passage; and
disinfecting the air is within the internal air passage by the UV light emitted by the one or more UV lights and reflected by the internal reflective surfaces.

Clause 23. A system comprising:
a duct including internal reflective surfaces surrounding at least a portion of an internal air passage, wherein the duct is a light pipe;
one or more ultraviolet (UV) lights coupled to the duct, wherein the one or more UV lights are configured to emit UV light into air that passes through the internal air passage, wherein the internal reflective surfaces reflect the UV light within the internal air passage;
an air inlet coupled to the duct, wherein the air inlet is in fluid communication with the internal air passage, wherein the air inlet has a first diameter;
an air outlet coupled to the duct, wherein the air outlet is in fluid communication with the internal air passage, wherein the air outlet comprises a nozzle having a second diameter, wherein the second diameter is less than the first diameter, wherein the nozzle is moveable, wherein the air inlet or the air outlet are formed of a UV absorbing material, wherein one or both of the air inlet or the air outlet comprises fins that are configured to absorb the UV light, and wherein one or both of the air inlet or the air outlet comprises a screen;
an outlet tube extending from the nozzle of the air outlet; and
a blower coupled to the duct, wherein the blower is configured to draw the air into the internal air passage through the air inlet, and discharge the air from the internal air passage through the air outlet, and wherein the air is disinfected within the internal air passage by the UV light emitted by the one or more UV lights and reflected by the internal reflective surfaces.

As described herein, examples of the present disclosure provide systems and methods for disinfecting air, such as within a confined space (for example, an internal cabin of a vehicle).

While various spatial and directional terms, such as top, bottom, lower, mid, lateral, horizontal, vertical, front and the like can be used to describe examples of the present disclosure, it is understood that such terms are merely used with respect to the orientations shown in the drawings. The orientations can be inverted, rotated, or otherwise changed, such that an upper portion is a lower portion, and vice versa, horizontal becomes vertical, and the like.

As used herein, a structure, limitation, or element that is “configured to” perform a task or operation is particularly structurally formed, constructed, or adapted in a manner corresponding to the task or operation. For purposes of clarity and the avoidance of doubt, an object that is merely capable of being modified to perform the task or operation is not “configured to” perform the task or operation as used herein.

It is to be understood that the above description is intended to be illustrative, and not restrictive. For example, the above-described examples (and/or aspects thereof) can be used in combination with each other. In addition, many modifications can be made to adapt a particular situation or material to the teachings of the various examples of the disclosure without departing from their scope. While the dimensions and types of materials described herein are intended to define the aspects of the various examples of the disclosure, the examples are by no means limiting and are exemplary examples. Many other examples will be apparent to those of skill in the art upon reviewing the above description. The scope of the various examples of the disclosure should, therefore, be determined with reference to the appended claims, along with the full scope of equivalents to which such claims are entitled. In the appended claims and the detailed description herein, the terms “including” and “in which” are used as the plain-English equivalents of the respective terms “comprising” and “wherein.” Moreover, the terms “first,” “second,” and “third,” etc. are used merely as labels, and are not intended to impose numerical requirements on their objects. Further, the limitations of the following claims are not written in means-plus-function format and are not intended to be interpreted based on 35 U.S.C. § 112(f), unless and until such claim limitations expressly use the phrase “means for” followed by a statement of function void of further structure.

This written description uses examples to disclose the various examples of the disclosure, including the best mode, and also to enable any person skilled in the art to practice the various examples of the disclosure, including making and using any devices or systems and performing any incorporated methods. The patentable scope of the various examples of the disclosure is defined by the claims, and can include other examples that occur to those skilled in the art. Such other examples are intended to be within the scope of the claims if the examples have structural elements that do not differ from the literal language of the claims, or if the examples include equivalent structural elements with insubstantial differences from the literal language of the claims.

What is claimed is:

1. A system comprising:
a duct including internal reflective surfaces surrounding at least a portion of an internal air passage;
one or more ultraviolet (UV) light emitters coupled to the duct, wherein the one or more UV light emitters are configured to emit UV light into air that passes through the internal air passage, wherein the internal reflective surfaces reflect the UV light within the internal air passage;
an air inlet coupled to the duct, wherein the air inlet is in fluid communication with the internal air passage;
an air outlet coupled to the duct, wherein the air outlet is in fluid communication with the internal air passage; and
a blower coupled to the duct, wherein the blower is configured to draw the air into the internal air passage through the air inlet, and discharge the air from the internal air passage through the air outlet, and wherein the air is disinfected within the internal air passage by the UV light emitted by the one or more UV light emitters and reflected by the internal reflective surfaces.

2. The system of claim 1, wherein the duct comprises a first segment connected to a second segment through a bend.

3. The system of claim 2, wherein the bend positions the air outlet proximate to the air inlet.

4. The system of claim 2, wherein the bend provides a 180 degree turn.

5. The system of claim 1, wherein the one or more UV light emitters are configured to emit the UV light at a wavelength ranging from 270-280 nanometers.

6. The system of claim 1, wherein the one or more UV light emitters are configured to emit the UV light at a wavelength ranging from 210-260 nanometers.

7. The system of claim 1, wherein one or both of the air inlet or the air outlet are formed of a UV absorbing material.

8. The system of claim 7, wherein the UV absorbing material is a dark plastic.

9. The system of claim 1, wherein the air outlet comprises a nozzle, wherein the air inlet has a first diameter, and the nozzle has a second diameter, and wherein the second diameter is less than the first diameter.

10. The system of claim 9, wherein the nozzle is moveable.

11. The system of claim 1, wherein the duct is a light pipe.

12. The system of claim 1, wherein the system is configured to be worn by an individual.

13. The system of claim 1, wherein the system is incorporated into a headrest of a seat.

14. The system of claim 13, wherein the system is moveable between a stowed position and a deployed position.

15. The system of claim 1, further comprising a mounting member configured to secure the system to a structure.

16. The system of claim 1, wherein one or both of the air inlet or the air outlet comprises fins that are configured to absorb the UV light.

17. The system of claim 1, further comprising an outlet tube extending from a nozzle of the air outlet.

18. The system of claim 17, wherein the outlet tube is removably secured to the nozzle.

19. The system of claim 17, wherein the outlet tube is pivotally coupled to the nozzle.

20. The system of claim 1, wherein one or both of the air inlet or the air outlet comprises a screen.

21. The system of claim 1, further comprising a flexible tube connected to the duct, wherein the flexible tube comprises the air outlet.

22. A method comprising:
emitting, from one or more ultraviolet (UV) light emitters coupled to a duct including internal reflective surfaces surrounding at least a portion of an internal air passage, UV light into air that passes through the internal air passage;
reflecting, by the internal reflective surfaces, the UV light within the internal air passage;
drawing, by a blower coupled to the duct, the air into the internal air passage through an air inlet coupled to the duct, wherein the air inlet is in fluid communication with the internal air passage;
discharging, by the blower the air from the internal air passage through an air outlet coupled to the duct, wherein the air outlet is in fluid communication with the internal air passage; and
disinfecting the air is within the internal air passage by the UV light emitted by the one or more UV light emitters and reflected by the internal reflective surfaces.

23. A system comprising:
a duct including internal reflective surfaces surrounding at least a portion of an internal air passage, wherein the duct is a light pipe;
one or more ultraviolet (UV) light emitters coupled to the duct, wherein the one or more UV light emitters are configured to emit UV light into air that passes through the internal air passage, wherein the internal reflective surfaces reflect the UV light within the internal air passage;
an air inlet coupled to the duct, wherein the air inlet is in fluid communication with the internal air passage, wherein the air inlet has a first diameter;
an air outlet coupled to the duct, wherein the air outlet is in fluid communication with the internal air passage, wherein the air outlet comprises a nozzle having a second diameter, wherein the second diameter is less than the first diameter, wherein the nozzle is moveable, wherein the air inlet or the air outlet are formed of a UV absorbing material, wherein one or both of the air inlet or the air outlet comprises fins that are configured to absorb the UV light, and wherein one or both of the air inlet or the air outlet comprises a screen;
an outlet tube extending from the nozzle of the air outlet; and
a blower coupled to the duct, wherein the blower is configured to draw the air into the internal air passage through the air inlet, and discharge the air from the internal air passage through the air outlet, and wherein the air is disinfected within the internal air passage by the UV light emitted by the one or more UV light emitters and reflected by the internal reflective surfaces.
